(19) United States
(12) Patent Application Publication
(10) Pub. No.: US 2020/0326393 A1
(43) Pub. Date: Oct. 15, 2020

(54) Shoulder Coil for Use in Magnetic Resonance Imaging

(71) Applicant: Neocoil, Inc., Pewaukee, WI (US)

(72) Inventors: Kyle Johnson, Brookfield, WI (US); Brian Brown, Wauwatosa, WI (US); Venkat Goruganti, Sussex, WI (US); Michael Haase, New Berlin, WI (US)

(21) Appl. No.: 16/381,496

(22) Filed: Apr. 11, 2019

(51) Int. Cl. G01R 33/3415; G01R 33/563; G01R 33/34; A61B 5/055

(52) U.S. Cl. CPC G01R 33/3415; A61B 5/0555; G01R 33/34084; G01R 33/563

(57) ABSTRACT

A shoulder coil for use in MR imaging includes a rigid housing configured to be positioned on the posterior portion of the shoulder and a flexible coil assembly, mounted to the rigid housing, where the flexible coil assembly is repositionable with respect to the first housing. The flexible coil assembly may be folded outward to facilitate loading of the patient or positioning of the shoulder coil with respect to the patient and folded inward to rest on the shoulder of the patient during imaging. The shoulder coil is positioned at an angle with respect to the shoulder to extend from the superior surface to the lateral surface of the shoulder. Antennas are located in the rigid housing, flexible coil assembly and between the two housings to detect NMR signals from each surface of the shoulder.

### BACKGROUND OF THE INVENTION

The subject matter disclosed herein relates to a system for use in obtaining a magnetic resonance (MR) image of the shoulder of a patient. More specifically, the assembly includes a rigid support structure with a first set of antenna arrays located therein to image a posterior portion of the shoulder and a flexible housing with a second set of antenna arrays located therein to be positioned on the anterior portion of the shoulder.

As is known to those skilled in the art, an MR system generates a strong magnetic field which aligns nuclei in the presence of the magnetic field and then detects the faint nuclear magnetic resonance (NMR) signals given off by nuclei returning to a normal state in the absence of the magnetic field. The NMR signals vary as a function of the type of organ, bone, tissue, etc. . . . present within the magnetic field. The NMR signals are received by antennas, also referred to as local coils, and transmitted to the MR scanner for reconstruction into an MR image. Specifically, an anatomical region of a patient is located within the magnetic field and proximate to the antennas. The MR scanner reconstructs the NMR signals into an MR image corresponding to the anatomical region of the patient being imaged.

In order to provide high quality images, it is desirable to uniformly detect the NMR signals across the anatomical region of interest. It is also desirable to obtain the NMR signals with a high signal-to-noise ratio (SNR). In order to detect the NMR signals across the anatomical region of interest, a receiving antenna must be proximate to the anatomical region to receive the NM R signals. A single antenna could be used that spans the entire anatomical region. However, as the size of the antenna increases, it becomes more susceptible to detecting noise and the SNR decreases. In order to increase the SNR, therefore, an array of smaller antennas, spaced out over the anatomical region of interest may be provided.

It is also known to provide a housing in which the array of antennas is placed to maintain a desired spacing or arrangement of the antennas with respect to each other and with respect to the anatomical region of interest. In some applications, the housing may be rigid while in other applications, the housing may be flexible. A rigid housing permits the antennas to be positioned precisely within the housing and the arrangement of the antennas to be maintained. The rigid housing, however, must be sized to accept the anatomy of the largest patient expected to be imaged. For the majority of patients, this sizing will result in some space between the anatomy to be imaged and the housing. As the distance between the antenna and the anatomy to be imaged increases, the magnitude of the signal detected by the antenna decreases, resulting in a lower SNR. To improve the SNR, it is desirable to position the antennas closer to the anatomical region being imaged. In contrast, a flexible housing permits the antennas to placed directly on the anatomical region being imaged. The flexible housing conforms to the contours of the anatomical region and the antennas within the flexible housing similarly follow the contours of the anatomical region being imaged. The flexible housing, however, may cause antennas to be positioned closer to or farther from adjacent antennas as the flexible substrate to which the antennas are mounted gathers together or spreads out. The flexible housing additionally must be placed in the proper orientation on the anatomical region being imaged and must be secured in position to prevent movement of the antennas during imaging.

For certain imaging applications, positioning the antennas with respect to the anatomical region to be imaged presents challenges. A shoulder of a patient, for example, is a complex structure extending from the rear, or posterior, to the front, or anterior, regions of a patient. It also is positioned at an upper, or superior, as well as a side, or lateral, portion of the arm. In order to obtain a comprehensive image of a shoulder, it is desirable to detect the NMR signals from each of the different directions with respect to the shoulder.

Another problem during shoulder imaging is the appearance of motion artifacts in the image obtained by the scanner. Motion artifacts are a result of either the patient moving or the antennas moving with respect to the patient during imaging. Patient motion may be caused by the expanding and contracting of the lungs during breathing occurring within the field of view of the region to be imaged or due to twitching or shifting of a patient as a result of discomfort or loss of attentiveness during the procedure. Antennas may move with respect to the patient if the housing in which the antennas are located move as a result of patient motion or due to slippage off a patient as a result of insecure fastening to the patient. A shoulder of a patient, for example, has many sloped surfaces from which the housing may fall. Thus, it would be desirable to stabilize the shoulder joint and to secure the antenna housing to the patient during imaging.

### BRIEF DESCRIPTION OF THE INVENTION

The subject matter disclosed herein describes a shoulder coil that detects NMR signals from a shoulder of a patient from each of the different directions with respect to the shoulder while achieving a high SNR. The shoulder coil includes a first housing that is rigid and configured to be positioned on the posterior portion of the shoulder. The shoulder coil also includes a flexible coil assembly mounted to the first housing, where the flexible coil assembly is repositionable with respect to the first housing. The flexible coil assembly may, for example, be folded outward from the first housing to facilitate loading of the patient and/or positioning of the shoulder coil with respect to the patient, and the flexible coil assembly may be folded inward to rest on the shoulder and/or chest of the patient during imaging. The shoulder coil is positioned at an angle with respect to the shoulder such that the shoulder coil extends around the shoulder from the superior surface to the lateral surface of the shoulder. Antennas located in the rigid housing detect NMR signals from the posterior surface of the shoulder, antennas located in the flexible housing detect NMR signals from the anterior surface of the shoulder, and antennas spanning between the rigid and flexible housings detect NMR signals from the span between the superior and lateral surfaces of the shoulder. A strap may be provided that extends from the rigid housing and around the shoulder to help stabilize the shoulder and to secure the shoulder coil to the shoulder during imaging.

According to one embodiment of the invention, a shoulder coil for use during medical imaging is disclosed. The shoulder coil includes a lower support member, a flexible coil assembly, multiple antennas, and a cable. The lower support member includes a rigid housing configured to be positioned between a shoulder of a patient and a table on which the patient is located and multiple first antennas are positioned within the rigid housing. The flexible coil assembly is mounted to the rigid housing and is movably positioned with respect to the rigid housing to allow the shoulder of the patient to be positioned on the rigid housing with the flexible coil assembly in a first position and to be placed adjacent to the shoulder of the patient with the flexible coil assembly in a second position. The flexible coil assembly includes multiple second antennas. Multiple third antennas extend between the lower support member and the flexible coil assembly, where the third antennas are located within at least one of the rigid housing and the flexible coil assembly and are configured to be positioned between a superior surface of the shoulder and a lateral surface of the shoulder during medical imaging. A cable extends from either the rigid housing or the flexible coil assembly to a magnetic resonance imaging (MRI) scanner, where the cable transmits a signal generated by each of the first antennas, second antennas, and third antennas to the MRI scanner.

According to another aspect of the invention, the rigid housing includes a substantially planar lower surface operative to set on an upper surface of the table, and the first antennas are positioned within the rigid housing such that each of the first antennas is generally parallel to the upper surface of the table when the rigid housing is set on the upper surface of the table.

According to yet another aspect of the invention, the rigid housing is configured to receive a posterior side of the shoulder and the flexible coil assembly is configured to be positioned on an anterior side of the shoulder.

According to still other aspects of the invention the rigid housing includes a first portion and a second portion. The first portion is generally planar and is configured to be positioned between the shoulder of the patient and the table, and the second portion extends away from the table for at least a portion of the lateral surface of the shoulder. The first portion includes a first edge configured to be located between the shoulder of the patient and the table and a second edge configured to be located beyond the lateral surface of the shoulder. The second portion includes a first edge joined to the second edge of the first portion proximate to the table and a second edge distal from the table, and the second portion is curved to extend between the superior surface of the shoulder and the lateral surface of the shoulder. The first portion of the rigid housing may also have a first thickness, tapered to a second thickness along at least a portion of the first edge of the first portion, where the second thickness is less than the first thickness.

According to yet another aspect of the invention, the flexible coil assembly may include a retaining member configured to retain the flexible coil in the second position. The flexible coil assembly extends for a first distance from the rigid housing onto the shoulder of the patient. The flexible coil assembly also includes an outer periphery having a first edge mounted to the rigid housing and a second edge distal from the rigid housing and located over the shoulder during medical imaging. The retaining member may be a stiffening bar extending for at least a portion of the first distance. Optionally, the flexible coil assembly further includes a weighting member along at least a portion of the second edge.

According to another embodiment of the invention, a shoulder coil for use during medical imaging of a patient is disclosed. The shoulder coil includes a rigid housing having a first portion and a second portion, where the first portion is configured to be positioned between a shoulder of the patient and a table of a magnetic resonance (MR) scanner, and the second portion extends from the first portion away from the table and is configured to be positioned between a superior surface of the shoulder and a lateral surface of the shoulder of the patient during medical imaging. A chamber is defined within the first and second portions. A flexible coil assembly is mounted to the second portion of the rigid housing. The flexible coil assembly includes an inner housing, an outer housing, and a flexible substrate mounted between the inner and outer housing. The flexible coil assembly is movably positioned with respect to the rigid housing to allow the shoulder of the patient to be positioned on the rigid housing with the flexible coil assembly in a first position and to be placed adjacent to the shoulder of the patient with the flexible coil assembly in a second position. Multiple antennas defining an antenna array are positioned within the shoulder coil. A first set of antennas are positioned within the chamber of the rigid housing, and a second set of antennas are mounted to the flexible substrate of the flexible coil assembly.

These and other advantages and features of the invention will become apparent to those skilled in the art from the detailed description and the accompanying drawings. It should be understood, however, that the detailed description and accompanying drawings, while indicating preferred embodiments of the present invention, are given by way of illustration and not of limitation. Many changes and modifications may be made within the scope of the present invention without departing from the spirit thereof, and the invention includes all such modifications.

### BRIEF DESCRIPTION OF THE DRAWINGS

Various exemplary embodiments of the subject matter disclosed herein are illustrated in the accompanying drawings in which like reference numerals represent like parts throughout, and in which:

FIG. 1 is a perspective view of a shoulder coil according to one embodiment of the invention;

FIG. 2 is a side elevation view of the shoulder coil of FIG. 1;

FIG. 3 is a front elevation view of the shoulder coil of FIG. 1;

FIG. 4 is a rear elevation view of a connector for the shoulder coil of FIG. 1;

FIG. 5 is a top plan view of the shoulder coil of FIG. 1 with the upper layers of the flexible housing removed;

FIG. 6 is a rear elevation view of the shoulder coil of FIG. 1 with the rear housing removed;

FIG. 7 is a bottom plan view of the shoulder coil of FIG. 1 with the lower housing removed;

FIG. 8 is a partial exploded view of the shoulder coil of FIG. 1;

FIG. 9 is a partial exploded view of the flexible coil assembly for the shoulder coil of FIG. 1;

FIG. 10 is a sectional view of the shoulder coil taken at 10-10 of FIG. 3;

FIG. 11 is perspective view of a shoulder coil according to another embodiment of the invention, illustrating a stiffening member inserted into the flexible coil assembly;

FIG. 12 is a top plan view of a shoulder coil according to another embodiment of the invention, illustrating a weighting member inserted into the flexible coil assembly;

FIG. 13 is a top plan view of a shoulder coil according to another embodiment of the invention, illustrating a removable weighting member placed on the flexible coil assembly;

FIG. 14 is a perspective view of the shoulder coil of FIG. 1 in use on a table for a MRI scanner in an exemplary environment;

FIG. 15 is a perspective view of a first portion of the antennas within the shoulder coil of FIG. 14 illustrating the orientation of the first portion of the antennas with respect to a patient and with respect to the axes of the MRI scanner of FIG. 13;

FIG. 16 is a perspective view of a second portion of the antennas within the shoulder coil of FIG. 14 illustrating the orientation of the second portion of the antennas with respect to a patient and with respect to the axes of the MR scanner of FIG. 13;

FIG. 17 is a perspective view of the shoulder coil of FIG. 1 with the flexible coil assembly folded back to assist in positioning a patient on shoulder coil

FIG. 18 is a perspective view of a shoulder coil according to another embodiment of the invention;

FIG. 19 is a perspective view of the shoulder coil of FIG. 18 positioned on a patient; and

FIG. 20 is a bottom plan view of the shoulder coil of FIG. 18.

In describing the various embodiments of the invention which are illustrated in the drawings, specific terminology will be resorted to for the sake of clarity. However, it is not intended that the invention be limited to the specific terms so selected and it is understood that each specific term includes all technical equivalents which operate in a similar manner to accomplish a similar purpose. For example, the word “connected,” “attached,” or terms similar thereto are often used. They are not limited to direct connection but include connection through other elements where such connection is recognized as being equivalent by those skilled in the art.

### DETAILED DESCRIPTION OF THE INVENTION

The various features and advantageous details of the subject matter disclosed herein are explained more fully with reference to the non-limiting embodiments described in detail in the following description.

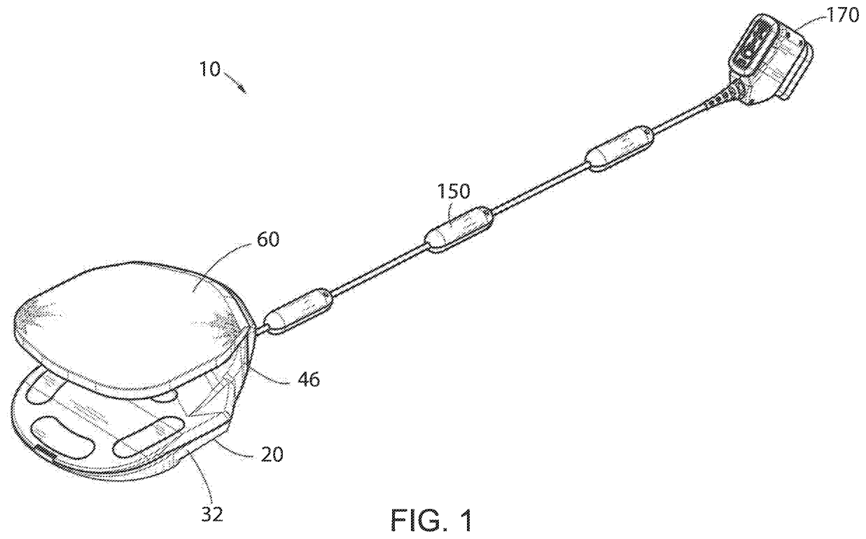

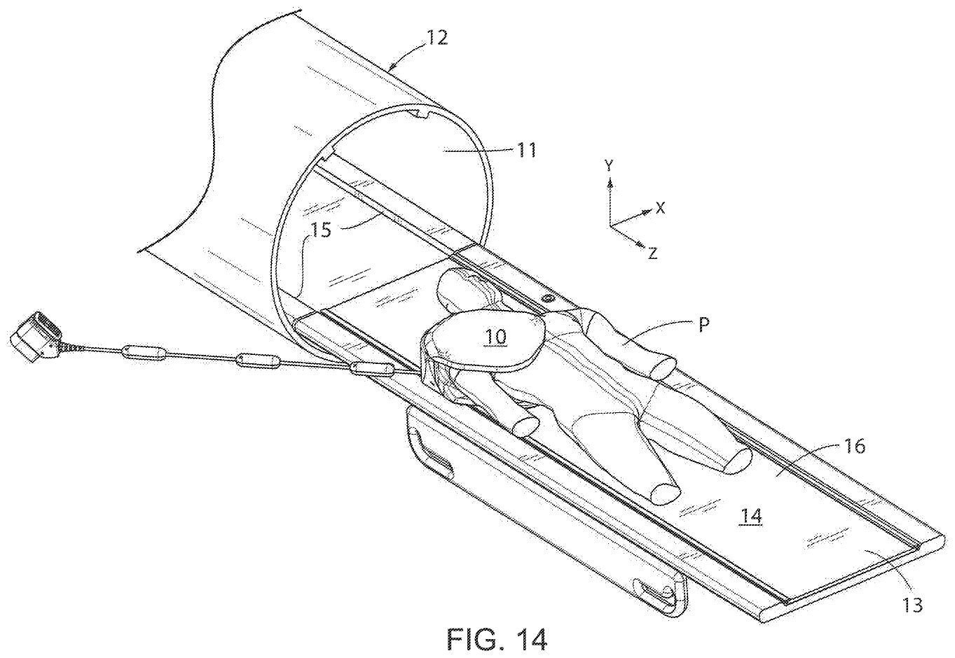

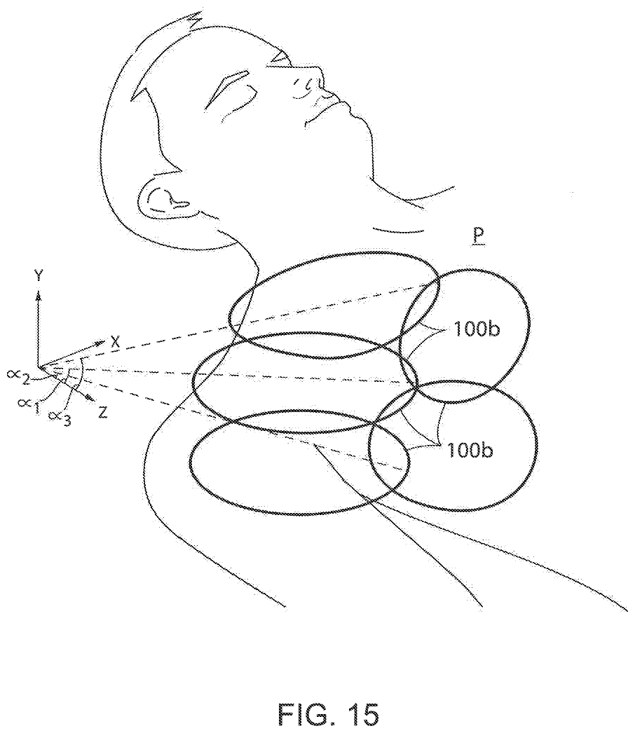

Turning initially to FIG. 1, one embodiment of a shoulder coil 10 for use in MR imaging is illustrated. The shoulder coil 10 includes a rigid housing 20 and a flexible coil assembly 60. The rigid housing 20 is configured to be positioned on an upper surface 16 of a table 14 for a MR scanner 12 (see also FIG. 14) and to orient the shoulder coil 10 with respect to a patient, P, lying on the table. The flexible coil assembly 60 may be folded back to an open position to assist with positioning the patient in the shoulder coil 10 and then folded forward to a closed position to rest on the anterior portion of the patient's shoulder. The combination of a rigid housing 20 as a first element of the shoulder coil 10 and a flexible coil assembly 60 as a second element of the shoulder coil provide the disclosed shoulder coil 10 with the benefits, while reducing the drawbacks, of each type of housing.

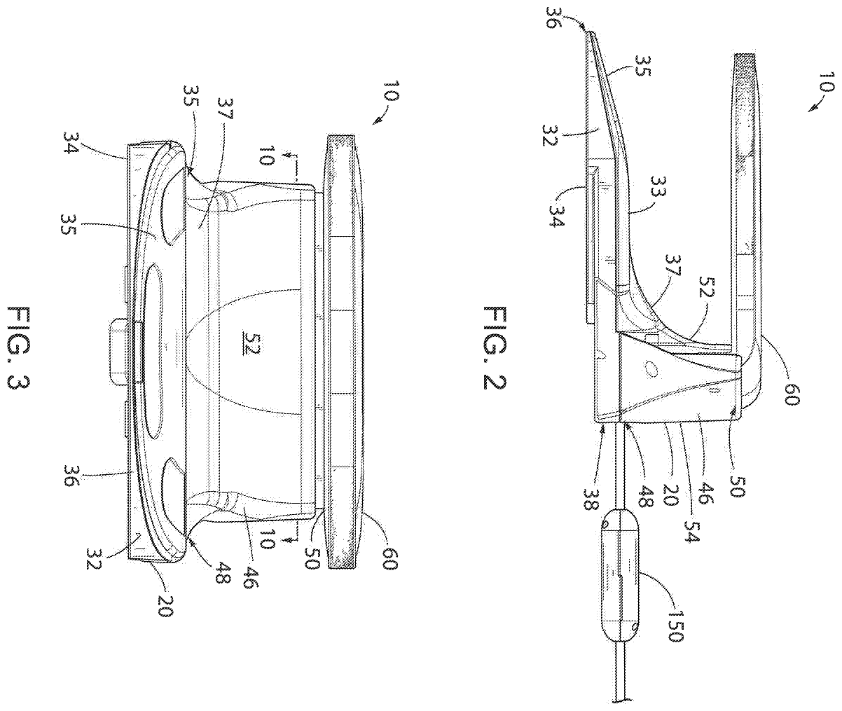

With reference also to FIGS. 2-3, the rigid housing 20 includes a first portion 32 and a second portion 46. The first portion 32 is generally planar and configured to be positioned between the patient, P, and the table 14 during imaging. The first portion 32 extends between a first edge 36 and a second edge 38, where the first edge 36 is located between the patient, P, and the table 14 while the second edge 38 is positioned beyond the lateral surface of the shoulder during imaging. A central region 33 of the first portion 32 has a first thickness and defines a chamber within of sufficient size to receive antennas and associated electronic components for the antennas within the housing. The central region 33 is generally planar and provides a level surface on which a posterior surface of the shoulder may be positioned. A tapered region 35 of the first portion 32 proximate the first edge 36 decreases from the first thickness at the central region 33 to a second thickness at the first edge 36, where the second thickness is less than the first thickness. The tapered region 35 maintains a thickness sufficient to include an antenna but does not need to include electronic components associated with the antennas. The tapered region 35 assists MR technicians, medical staff, or other trained personnel with insertion of the shoulder coil 10 under a shoulder of a patient while the patient is lying on the table 14. A sloped region 37 of the first portion 32 is proximate the second edge 38 and defines a curved surface transitioning, or beginning to transition, from the planar surface of the central region 33 to an upright surface extending upward along the lateral surface of the shoulder.

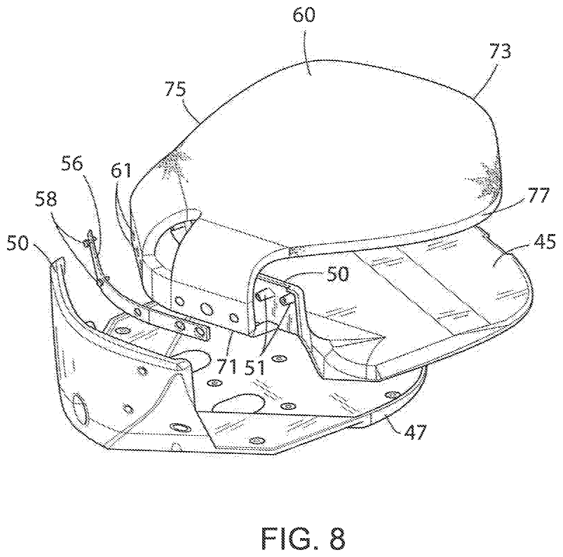

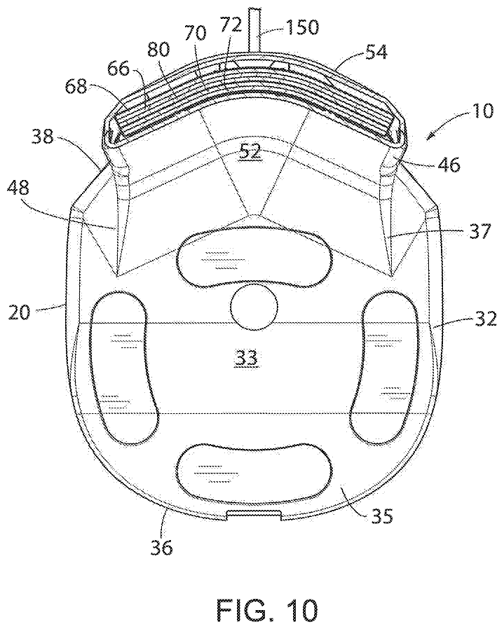

The second portion 46 of the rigid housing is configured to extend generally upward from the first portion 32 adjacent to the shoulder of the patient. The second portion 46 includes a first edge 48 and a second edge 50. The first edge 48 of the second portion 46 is configured to join the second edge 38 of the first portion 32. The second portion 46 has a curved interior surface 52 (see also FIG. 10) configured to wrap around the shoulder. The second portion 46 additionally defines a chamber within the interior and exterior surfaces 52, 54. The chamber is configured to hold antennas and associated electronic components. The chamber may further include an interface to a cable 150 by which signals from each of the antennas are transmitted to the MR scanner 12. The chamber in the second portion 46 is in air communication with the chamber in the first portion 32 such that a continuous chamber exists within the rigid housing 20. It is contemplated that the first portion 32 and the second portion 46 may be manufactured as separate elements, a single element, or alternately, as an interior rigid housing 45 and an exterior rigid housing 47 (see FIG. 8). The description herein of a first portion 32 and a second portion 46 is not limiting to two physical structures mounted together, but rather, to generally describe the rigid housing 20, which may be assembled from various different components into the form described herein.

With reference next to FIG. 8, the flexible coil assembly 60 is mounted within an opening along the second edge 50 of the second portion 46 of the rigid housing 20. According to the illustrated embodiment, an inner housing 45 and an outer housing 47 define the two portions 32, 46 of the rigid housing 20. Bosses 51 protrude from the inner housing 45 toward the outer housing 47 along the second edge 50. Openings 61 in the flexible coil assembly 60 are configured to fit over the bosses. A mounting bracket 56 is, in turn fit over the outer layer of the flexible coil assembly and fasteners 58 engage the bosses 51 to positively retain the mounting bracket and the flexible coil assembly 60 to the inner housing 45. According to one embodiment of the invention, the fasteners 58 may be threaded, such as screws or bolts, and engage a threaded interior of the bosses 51. According to another embodiment of the invention, the fasteners 58 may include a tab to engage a complementary member on the boss 51. According to still another embodiment of the invention, the fasteners 58 may be retained via a friction fit. The outer housing 47 is then mounted to the inner housing 45 to form the rigid housing 20 and enclose the mounting area for the flexible coil assembly 60.

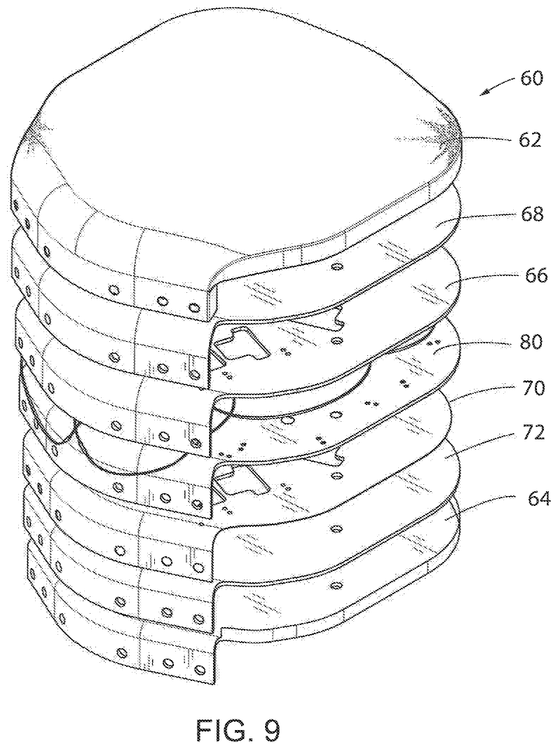

With reference next to FIG. 9, one embodiment of the flexible coil assembly 60 is illustrated. The flexible coil assembly 60 includes multiple layers of flexible material allowing the flexible coil assembly to conform to an anatomical region to be imaged. According to the illustrated embodiment, the flexible coil assembly 60 includes an outer housing 62 and an inner housing 64. The outer and inner housings 62, 64 are made of a pliable material suitable for magnetic resonance imaging, such as non-magnetic materials and materials that do not create proton signals during imaging. One such exemplary material is Dartex®. The material is preferably durable to withstand abrasion or contact with other surfaces and is preferably easily cleaned, for example, by wiping the outer surfaces with a disinfectant cleaner between uses. A flexible substrate 80 is provided between the outer and inner housings. The flexible substrate 80 is configured to have antennas and their associated electronic components mounted to the flexible substrate 80. The flexible substrate 80 is preferably a dimensionally stable fabric material, resisting stretching or shrinking; abrasion resistant to avoid damage by electronic components and/or circuit boards mounted to the substrate; and heat resistant, to avoid damage should localized heating occur during medical imaging. The flexbile substrate 80 may include, for example, Kevlar®, Nomex®, or Basofil® fibers, or a combination thereof. The flexible coil assembly 60 further includes a first padded layer 66 and a second padded layer 68 located between the flexible substrate 80 and the outer housing 62 as well as a third padded layer 70 and a fourth padded layer 72 located between the flexible substrate 80 and the inner housing 64. Each of the padded layers may be, for example, a foam material, a felt material, or other woven material. The padded layers serve to both protect the antennas and electronic components mounted to the flexible substrate 80 and to provide patient comfort when the flexible coil assembly 60 is positioned on the shoulder for imaging. Optionally, a single padded layer, layers having different composition, additional layers, or a combination thereof may be provided.

The outer housing 62 and the inner housing 64 of the flexible coil assembly 60 are joined together to define a housing closed around the periphery except for an opening 79 located along a first edge 71 of the flexible coil assembly 60 retained within the rigid housing 20. The flexible coil assembly extends for a length between the first edge 71 and a second edge 73 opposite the first edge, where the second edge is positioned over the anatomical region to be imaged during imaging. The flexible coil assembly 60 further spans between a first side 75 and a second side 77 opposite the first side, where each of the first and second sides 75, 77 extend between the first edge 71 and the second edge 73. It is further contemplated that each of the first edge 71, second edge 73, first side 75, and second side 77 may be straight, curved, or include multiple segments to define a flexible coil assembly 60 having any desired shape. According to the illustrated embodiment, the first edge 71 is generally straight and configured for insertion within the rigid housing 20 while the second edge 73 is curved. Each of the first and second sides 75, 77 includes multiple segments. A first segment extends from the first edge 71 away from the rigid housing 20 at an angle, such that that the width between the first and second sides increases as the flexible coil assembly 60 extends away from the rigid housing 20. After extending away from the rigid housing for a first distance, each of the first and second sides 75, 77 includes a second segment, where the second segments extend generally parallel to each other, such that the width of the flexible coil assembly is generally constant between the second segments of the first and second sides 75, 77.

Referring next to FIGS. 5-7, the shoulder coil 10 includes antennas 100 positioned throughout the shoulder coil 10 to detect the NMR signals from various angles around the shoulder of a patient. Each antenna 100 is a conductive loop connected to an electronic circuit. The electronic circuit is a pick-up circuit that converts the NMR signals detected by the conductive loop to an electronic signal deliverable to the MR scanner 12. Preferably, one pick-up circuit exists for each antenna 100. However, it is contemplated that a single circuit may include multiple channels, receiving signals from multiple antennas 100. Pick-up circuits may be mounted to a circuit board substrate which is, in turn, mounted to the flexible substrate 80 or within the rigid housing 20. When mounted to the flexible substrate 80, the circuit board substrate may be a rigid material if the substrate does not significantly impede the flexible coil assembly 60 from being positioned on the shoulder. Alternately, the circuit board substrate may be a flexible substrate, to aid the flexible housing 60 in conforming to the contours of the anatomy being image. According to one embodiment of the invention, a pre-amplifier circuit may be included in whole or in part on the circuit board supporting each pick-up circuit. Alternately, conductors from each pick-up circuit may extend to a separate pre-amplifier circuit or pre-amplifier module. The pre-amplifier circuit may be mounted, for example, on a rigid circuit board substrate within the rigid housing 20 of the shoulder coil 10. According to one embodiment of the invention, the pre-amplifier circuit may be housed external to the shoulder coil 10. A conductor is connected from each pick-up or pre-amplifier circuit to transmit the NMR signals received by each antenna 100 to the MR scanner 12.

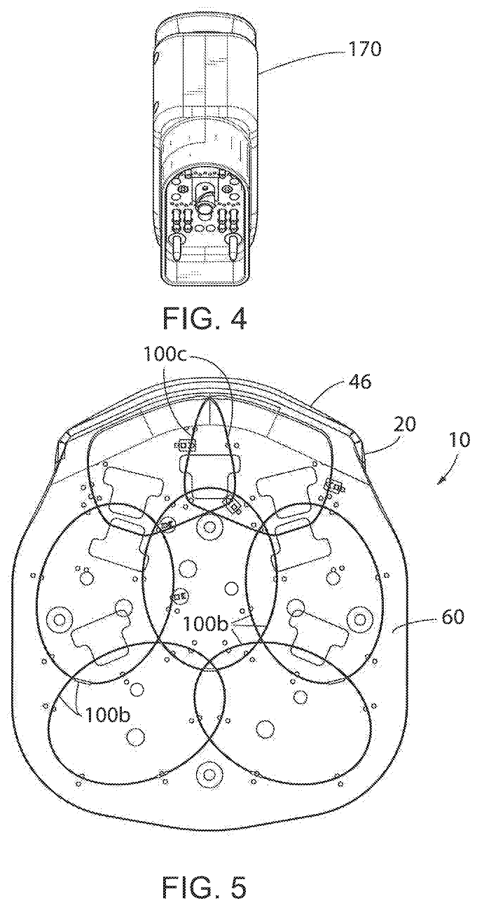

All of the conductors may be bundled into a cable 150 that is pre-terminated to a connector 170 configured to plug into the MR scanner 12. At least one cable 150 is connected to the shoulder coil 10. Each cable 150 includes at least one conductor carrying the signals from each antenna 100. The cable(s) 150 are preferably pre-terminated to a single connector 170 (see, for example, FIG. 4), such that the shoulder coil 10 may be quickly connected or disconnected as a single unit to the MR scanner 12.

In operation, the shoulder coil 10 is positioned proximate a patient's shoulder to provide signals from which the MR scanner 12 can generate a magnetic resonance image (MRI). With reference to FIGS. 14-17, a patient, P, is positioned on a table 14 for the MR scanner 12. A central portion 13 of the table 14 is configured to slide into and out of a bore 11 for the MR scanner 12. Guides, or rails, 15 may be positioned along the slide on a surface of the table between the central portion 13 and the rest of the table, directing the central portion 13 and the patient positioned on the central portion to move into and out of the bore 11.

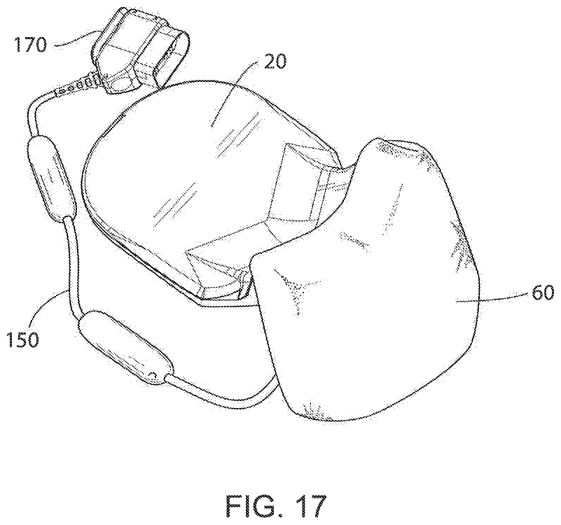

As shown in FIG. 17, the flexible coil assembly 60 is folded back into a loading position. The shoulder coil 10 may be placed on the table 14 prior to the patient, P, being positioned on the table 14. The patient, P, may lay back onto the first portion 32 of the rigid housing 20 such that the posterior surface of the patient's shoulder is located on the rigid housing 20. A technician or other trained medical personnel may adjust the shoulder coil 10 to ensure that it is aligned with the patient's shoulder to obtain the desired image. The flexible coil assembly 60 is folded forward over the anterior surface of the patient's shoulder and allowed to rest on the patient, conforming to the surface of the patient's shoulder.

Optionally, the patient may be loaded onto the table 14 prior to positioning the shoulder coil 10 on the table. The flexible coil assembly 60 may be folded back partially or entirely to a loading position. The tapered region 35 of the rigid housing 20 may then be slid under the posterior surface of the patient's shoulder. The tapered region 35 facilitates insertion of the shoulder coil 10 under the patient, P, when the patient is on the table 14 prior to positioning of the shoulder coil 10. The reduced thickness at the first edge 36 of the first portion 32 of the rigid housing 20 allows the shoulder coil 10 to be slid under the shoulder. The slope of the tapered region 35 toward the central region 33 as well as a slope to either side of the shoulder coil 10 help align the shoulder coil 10 with respect to the shoulder for imaging. After inserting the shoulder coil 10 under the patient's shoulder, the flexible coil assembly 60 is folded forward over the anterior surface of the patient's shoulder and allowed to rest on the patient, conforming to the surface of the patient's shoulder.

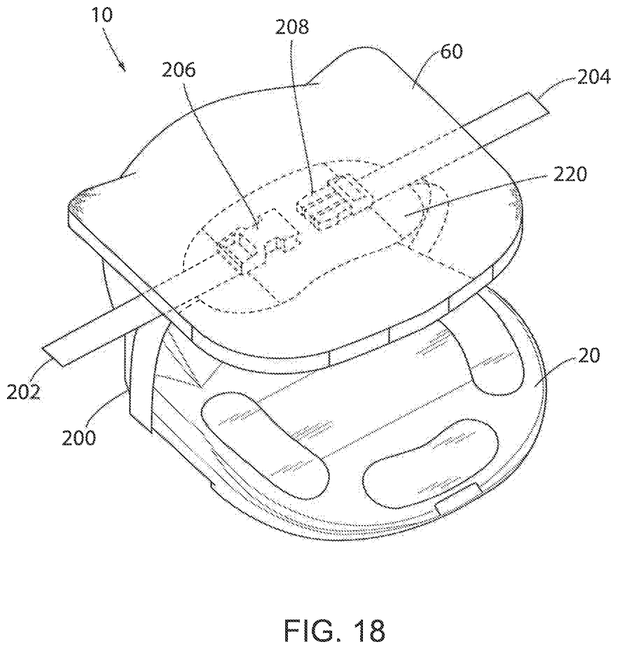

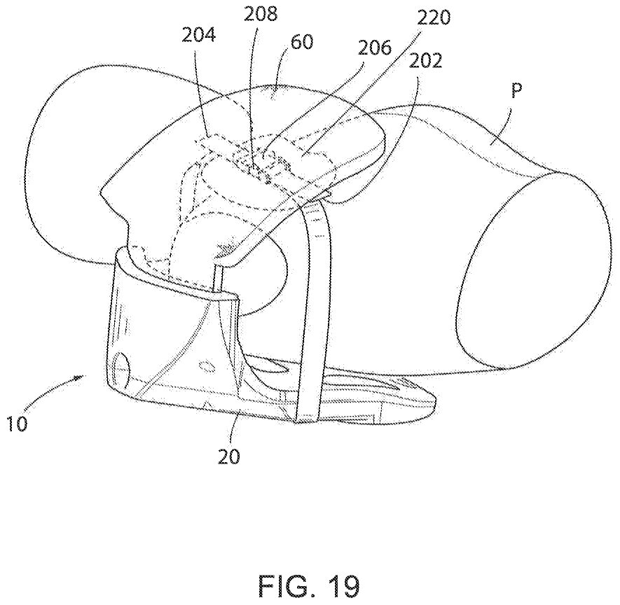

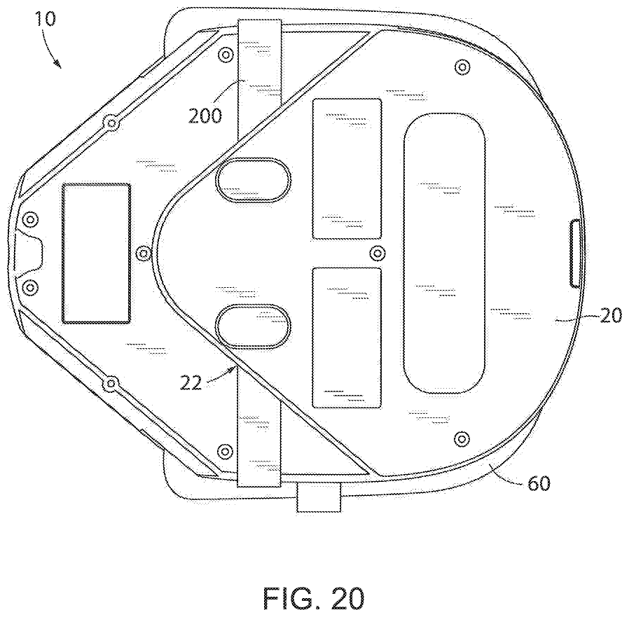

Referring also to FIGS. 18-20, a strap 200 may be included on the shoulder coil 10 to secure the shoulder coil 10 to the patient, P, and to stabilize the shoulder during imaging. As shown in FIG. 20, a bottom surface of the rigid housing 20 may include a slot 22 through which the strap 200 is passed. A first end 202 of the strap 200 extends beyond one side of the shoulder coil 10 and a second end 204 of the strap extends beyond the opposite side of the shoulder coil. When the shoulder coil 10 is on the imaging table and after the patient, P, has been positioned on the rigid housing 20, the ends of the strap 200 may be wrapped up around the anterior portion of the shoulder and joined together to secure the shoulder to the rigid housing 20. According to the illustrated embodiment a side release buckle is used to releasably connect the two ends of the strap 200. The first end 202 of the strap is fed through a first adjustment member on the receiving member 206 of the buckle, and the second end 204 of the strap is fed through a second adjustment member on the insertion member 208 of the buckle. The length of the strap 200 is adjustable by pulling on one or both ends 202, 204 of the strap to adjust the position of the receiving member 206, the insertion member 208 or both members of the buckle with respect to the strap 200. Optionally, a pad 220 may be provided and positioned on the shoulder of the patient, P, between the shoulder and the buckle to enhance the comfort of the patient. The pad 220 may be a pliable material having a greater surface area than the strap to distribute the pressure applied by the strap over a greater surface of the patient. The pad 220 may also include a cushion layer to provide stability of the shoulder while reducing the pressure felt by the patient as a result of the strap 200.

It is further contemplated that pad 220 may be secured to at least one of the buckle members such that the strap, buckle, and pad form a single assembly by which the shoulder coil 10 may be secured to the shoulder coil 10 of the patient, P, or to stabilize the shoulder during imaging. According to other embodiments of the invention, it is contemplated that the two ends of the strap may be secured to each other using various other fastening methods, including, but not limited to a hook and loop fastener or a button and stem fastener where at least a portion of the length of the strap proximate each end 202, 204 of the strap 200 includes one half of the hook and loop or button and stem fastener. During imaging, the MR scanner 12 generates a strong static magnetic field, B₀, (e.g., in the range of 1.5T to 7T) which causes the nuclei of atoms within the anatomical region being imaged to align with the magnetic field, B₀. The MR scanner 12 then transmits radio frequency (RF) magnetic field pulses transverse to B₀, where the transverse pulses are identified as B₁, to excite hydrogen atoms in the anatomical region to be imaged. Following the B₁ excitation, the atoms return to a normal state emitting NMR signals which are detected by the antennas 100 within the shoulder coil 10.

With reference to the axes defined in FIG. 14, the MR scanner 12 generates the static magnetic field, B₀, generally along the Z axis and generates the RF pulses, BI, transverse to B₀, generally in the X-Y plane. Because relaxation of the atoms emits magnetic signals in a direction orthogonal to the static magnetic field, B₀, detection of these signals occurs best when the magnetic fields of the coil antennas 100 align with the X-Y plane. An axis of each coil antenna 100 is defined as passing through the center of the loop defined by the antenna and generally perpendicular to the plane in which the antenna 100 is positioned. The axes of the antennas 100, therefore, are preferably aligned perpendicular to the axis of the static magnetic field, B₀, to obtain the highest magnitude signal. If the static magnetic field, B₀, extends generally in the Z axis, it is, therefore, desirable to have the axes of the antennas aligned in either the X axis or in the Y axis. Antennas 100 with axes oriented along the X axis or Y axis of the scanner 12 receive the strongest signal, and antennas 100 with axes oriented along the Z axis do not receive signals. Correspondingly, the antenna loops, as illustrated are preferably positioned within either the X-Z plane (i.e., axis of the antenna 100 oriented parallel to the Y axis) or the Y-Z plane (i.e., axis of the antenna 100 oriented parallel to the X axis). Antennas 100 positioned at angles in between will detect a portion of the signal and generate signals having a magnitude less than those positioned such that their axes are aligned in parallel with either the X or Y axis of the scanner where a full magnitude signal is detected.

As previously discussed, the shoulder is a difficult anatomical region from which to obtain an MR image. To obtain a complete image of the shoulder, it is desirable to detect the NMR signals emitted from the shoulder from multiple directions. Preferably, antennas would be located on the anterior, posterior, lateral and superior regions of the shoulder. However, an antenna 100 located along the superior surface of the shoulder lies generally in the X-Y plane of the MR scanner 12 and has its axis aligned with the static magnetic field, B₀. As a result, an antenna located along the superior surface of the shoulder will detect little or no signal during imaging.

As shown in FIG. 14, the present shoulder coil 10 is configured to align to the patient's shoulder such that the second portion 46 of the rigid housing 20 extends upward from the table 14 when the shoulder coil 10 is positioned at the shoulder and that the second portion 46 of the rigid housing is oriented approximately midway between the X-Y plane and the Y-Z plane of the MR scanner 12. It follows that the first portion 32 of the rigid housing 20 and the flexible coil assembly 60, which each attach to or are integrally formed with the second portion 46 of the rigid housing are similarly oriented approximately midway between the X-Y plane and the Y-Z plane of the MR scanner 12.

With reference again to FIGS. 5-7, the shoulder coil 10 includes three sets of antennas 100a, 100b, and 100c. The different sets of antennas are located throughout the shoulder coil 10 and configured to be oriented in various directions with respect to the patient's shoulder to obtain an image of the shoulder from each orientation. The first antennas 100a are located within the first portion 32 of the rigid housing 20. When the shoulder coil 10 is positioned on the table 14 of the MR scanner 12, each of the first antennas 100a are generally parallel to the table 14 and oriented within the X-Z plane of the MR scanner 12. The second antennas 100b are located within the flexible coil assembly 60. When the flexible coil assembly 60 is positioned on the anterior surface of the shoulder, each of the second antennas 100b follow the contour of the shoulder and or chest region on which the flexible coil assembly 60 is positioned. The third antennas 100c are located in part within the rigid housing 20 and in part within the flexible coil assembly 60. Two of the third antennas 100 are located primarily within the first portion 32 of the rigid housing 20 and wrap around the bend proximate the second edge 38 of the first portion 32 into the second portion 46 of the rigid housing 20. Third and fourth antennas, identified as part of the set of third antennas 100c, are positioned entirely within the second portion 46 of the rigid housing 20. The third and fourth antennas extend laterally from each side of the second portion 46 into the center of the second portion. Each of the third and fourth antennas follow the curvature of the curved interior surface 52 of the second portion such that a portion of each antenna 100c is positioned more along either the superior or lateral surface of the shoulder and a portion of each antenna 100c is positioned near the midpoint between the superior and lateral surfaces of the shoulder. A fifth and a sixth antenna, identified as part of the set of third antennas 100c, are located primarily within the flexible coil assembly 60. The fifth and sixth antennas are mounted to the flexible substrate 80 and a portion of each of the fifth and sixth antennas extend outward beyond the outer and inner housings 62, 64 of the flexible substrate 80 and into the rigid housing 20. As the flexible coil assembly 60 is positioned forward or backward, the fifth and sixth antennas 100c are similarly positioned within the assembly. Although discussed herein as three sets of antennas 100a, 100b, 100c, it is understood that the combination of all three sets of antennas forms a single antenna array. The signals from each of the three sets 100a, 100b, 100c are provided to the MR scanner 12 via the cable 150 and the M R scanner 12 can recreate an MR image based on the signals provided by the entire antenna array. The division into three sets of antennas is done for convenience of description and it is contemplated that the antennas 100 may be considered as a single set, two sets, individual antennas or in any other suitable arrangement.

The antennas 100 in the shoulder coil 10 can detect NMR signals from both the superior surface and the lateral surface of the shoulder. With reference also to FIG. 15, an exemplary arrangement of the coils 100b in the flexible coil assembly 60 is illustrated. A first antenna 100b is positioned at a first angle, α₁, that is about forty-five degrees (45°) between the X and Z axes. A second antenna 100b is positioned at a second angle, α₂, that is about thirty degrees (30°) displaced from the Z axis toward the X axis, and a third antenna 100b is positioned at a third angle, α₃, that is about sixty degrees (60°) displaced from the Z axis toward the X axis. The illustrated antennas 100b define, at least in part, the second set of antennas 100b within the flexible coil assembly 60. Antennas 100 may be similarly arranged at differing angles between the X-Y plane and the Y-Z plane within the first set of antennas 100a located in the first portion 32 of the rigid housing 20 and within the third set of antennas 100c spanning between the rigid housing 20 and the flexible coil assembly 60.

As previously discussed, antennas 100 with an axis orthogonal to the static magnetic field, B₀, receive the strongest signals from the anatomical regions being imaged. Thus, the antennas 100a in the rigid housing 20 and the antennas 100b in the flexible housing have axis generally parallel to the Y axis during imaging. The orientation of the shoulder coil 10 about the shoulder of the patient does not change the orientation of these axes with respect to the static magnetic field, B₀, and, therefore, these antennas 100a, 100b are able to detect strong signals from the region being imaged and, similarly provide signals with a high signal strength to the scanner 12. In contrast, the antennas 100c that either partially or fully extend in the vertical direction, or in the Y axis,

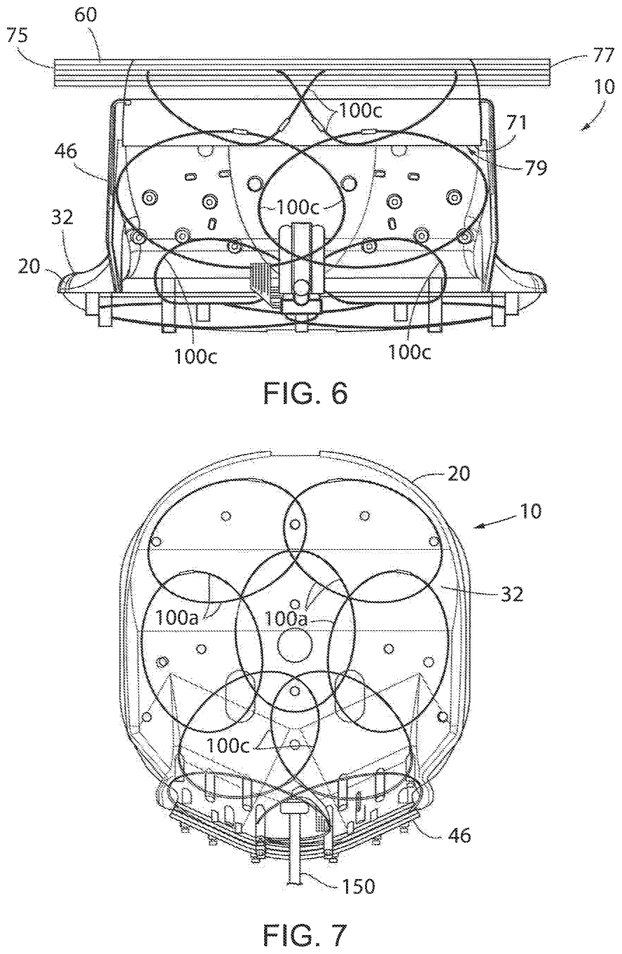

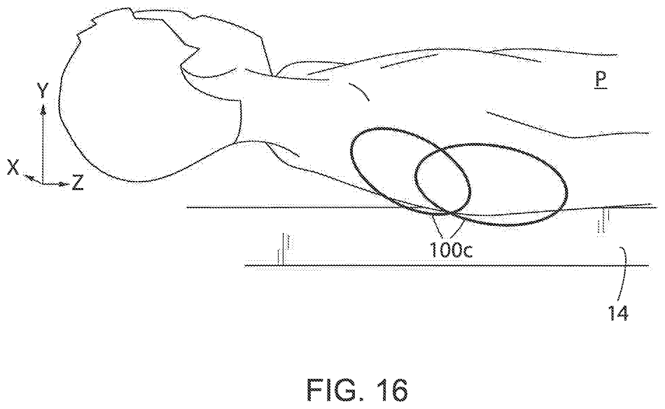

With reference to FIGS. 6 and 16, the third antennas 100c, as discussed above, include a vertical component, as they extend at least partially into the Y axis at the second edge 38 of the first portion 32 of the rigid housing 20, as they extend partially into the Y axis from the flexible coil assembly 60 as the flexible coil assembly 60 rests on the patient, or may be generally positioned in a vertical orientation as the antennas 100c span between the rigid housing 20 and the flexible coil assembly 60. Any of the vertical, or Y axis components located entirely within the X-Y plane have an axis extending along the Z-axis and, therefore, would detect no signal from the anatomical region being imaged. However, as the third antennas 100c are oriented further from the X-Y plane and closer to the Y-Z plane, the axis of the antenna becomes more orthogonal to the static magnetic field, B₀, and, therefore, the strength of the signal detected by the third antennas 100c increases as they are oriented further from the X-Y plane and closer to the Y-Z plane. Due to the orientation of the shoulder coil 10 with respect to the patient, P, each of the third antennas 100c with at least a portion of the antenna oriented in the vertical direction, these antennas 100c are oriented at an angle between the X-Y and the Y-Z planes and are not located directly superior to the shoulder in the X-Y plane. These third antennas 100c are located between the lateral and superior surfaces of the shoulder, and each of the vertical portions of the antennas 100 detects at least a portion of the signals emitted from the shoulder during imaging. The antennas 100c are able to detect a portion of the NMR signals from the superior surface and generate a signal for the scanner 12 corresponding to the detected signals, albeit at a reduced magnitude to an antenna fully orthogonal to the static magnetic field, BO.

Because the lateral face of the shoulder coil 10 is configured to align with the shoulder approximately midway between the X-Y plane and the Y-Z plane of the MR scanner 12, the shoulder coil 10 may be oriented on either shoulder of the patient, P, by rotating the shoulder coil 10 with respect to the patient and positioning the shoulder on or sliding the shoulder coil under the patient's shoulder. The portion of the shoulder coil 10 that receives NMR signals from the lateral surface of the right shoulder of a patient receives NMR signals from the superior surface of the left shoulder of the patient. Similarly, the portion of the shoulder coil 10 that receives NMR signals from the superior surface of the right shoulder of a patient receives NMR signals from the lateral surface of the left shoulder of the patient.

Although the flexible coil assembly 60 is illustrated as being generally horizontal when in position for imaging, the anterior surface of the shoulder and chest of a patient, P, is typically sloped upward from the lateral surface of the shoulder to the central region of the patient's chest. Consequently, the flexible coil assembly 60 similarly rests on the sloped surface rather than on a horizontal surface. Due to the sloped surface and motion of the patient resulting, for example, from respiration, the flexible coil assembly 60 may be prone to sliding down the sloped surface of the patient.

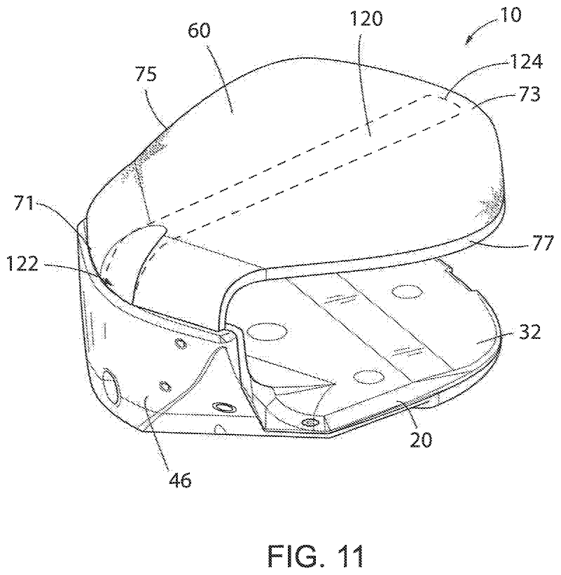

Referring next to FIGS. 11-13, the flexible coil assembly 60 may include a retaining member to resist motion of the flexible coil assembly 60 when it is positioned on the anterior surface of the patient's shoulder. With reference to FIG. 11, a first embodiment of the retaining member is a stiffening bar 120. The stiffening bar 120 includes a first end 122 and a second end 124. The first end 122 of the stiffening bar 120 is located proximate the first edge 71 of the flexible coil assembly 60, and the second end 124 of the stiffening bar 120 is located proximate the second edge 73 of the flexible coil assembly 60. The stiffening bar 120 is configured of a material that is flexible to allow the stiffening bar 120 to curl, or to be folded, up or down around an axis extending laterally to the bar when a force greater than the weight of the flexible coil assembly 60 is applied. The material, however, has sufficient stiffness to resistant bending solely from the weight of the flexible coil assembly attempting to slide off of the patient. Consequently, a technician or trained medical personnel can form the stiffening bar 120 to allow the flexible coil assembly 60 to be positioned, either away from the patient to facilitate positioning of the shoulder coil 10 or proximate the patient for imaging, yet the stiffening bar 120 maintains the position of the flexible coil assembly 60 proximate the patient once positioned, preventing the flexible coil assembly 60 from sliding off the anterior surface of the shoulder and/or the chest of the patient, P.

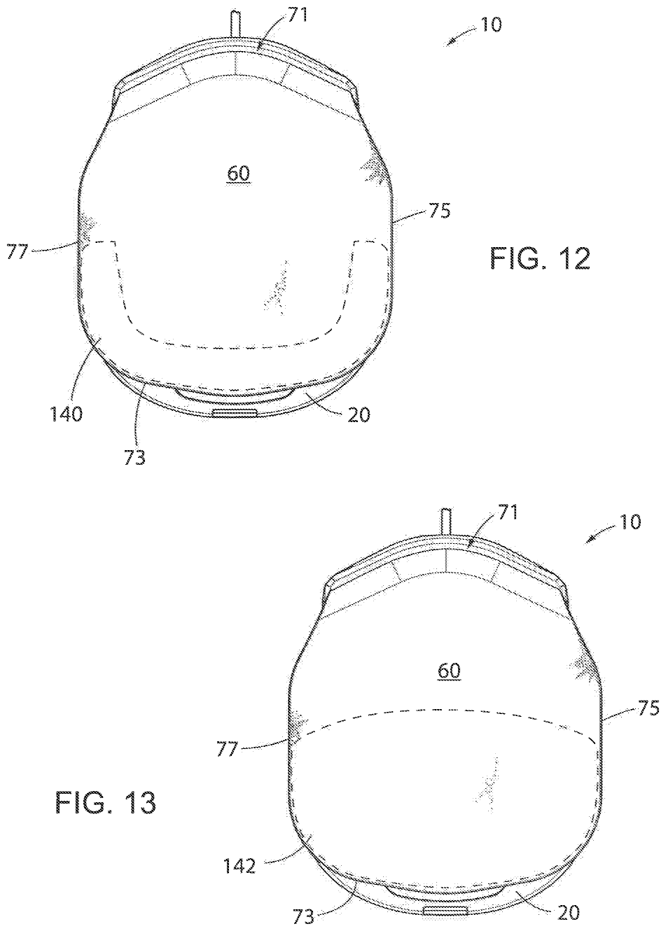

With reference to FIG. 12, a second embodiment of the retaining member is illustrated. A weighting member 140 is positioned between the outer housing 62 and the inner housing 64. The weighting member 140 is located along the periphery of the second edge 73 and may extend around for a portion of each side 75, 77. The weighting member 140 may be, for example, a fabric pouch with a fill material, such as plastic pellets, sand, or the like. The fill material is preferably a soft material having a small diameter such that the weighting member 140 may conform to the anatomical region being imaged. When the flexible coil assembly 60 is positioned on the shoulder and/or chest region of the patient, the weighting member 140 prevents the flexible coil assembly 60 from sliding off the anterior surface of the shoulder and/or the chest of the patient, P, and provides improved conformance of the flexible coil assembly 60 to the anatomy of the patient being imaged. Additionally, the weighting member 140 may assist in stabilizing the patient's shoulder and inhibit motion. The weighting member 140 may be used separately or in combination with the strap 200 discussed above to stabilize and immobilize the patient's shoulder. With reference also to FIG. 13, an alternate weighting member 142 may be removably positioned on the flexible coil assembly 60. It is contemplated that the alternate weighting member 142 may be used independently of the stiffening bar 120 or the weighting member 140 illustrated in FIG. 12. Optionally, the alternate weighting member 142 may be used in combination with the stiffening bar 120 or the weighting member 140 illustrated in FIG. 12. The alternate weighting member 142 may similarly be a pliable pouch filled with a fill material, such as plastic pellets, sand, or the like. The fill material is preferably a soft material having a small diameter such that the alternate weighting member 142 may conform to the anatomical region being imaged.

It should be understood that the invention is not limited in its application to the details of construction and arrangements of the components set forth herein. The invention is capable of other embodiments and of being practiced or carried out in various ways. Variations and modifications of the foregoing are within the scope of the present invention. It also being understood that the invention disclosed and defined herein extends to all alternative combinations of two or more of the individual features mentioned or evident from the text and/or drawings. All of these different combinations constitute various alternative aspects of the present invention. The embodiments described herein explain the best modes known for practicing the invention and will enable others skilled in the art to utilize the invention.

We claim:

1. A shoulder coil for use during medical imaging, comprising:
a lower support member including:
a rigid housing configured to be positioned between a shoulder of a patient and a table on which the patient is located, and
a plurality of first antennas positioned within the rigid housing;
a flexible coil assembly mounted to the rigid housing, wherein:
the flexible coil assembly is movably positioned with respect to the rigid housing to allow the shoulder of the patient to be positioned on the rigid housing in a first position and to be placed adjacent to the shoulder of the patient in a second position, and
the flexible coil assembly includes a plurality of second antennas;
a plurality of third antennas extending between the lower support member and the flexible coil assembly, wherein:
the plurality of third antennas are located within at least one of the rigid housing and the flexible coil assembly, and
the plurality of third antennas are configured to be positioned between a superior surface of the shoulder and a lateral surface of the shoulder during medical imaging; and
a cable extending from one of the rigid housing and the flexible coil assembly to a magnetic resonance imaging (MRI) scanner, wherein the cable transmits a signal generated by each of the first antennas, second antennas, and third antennas to the MRI scanner.

2. The shoulder coil of claim 1 wherein the rigid housing includes a substantially planar lower surface operative to set on an upper surface of the table and wherein the plurality of first antennas are positioned within the rigid housing such that each of the plurality of first antennas is generally parallel to the upper surface of the table when the rigid housing is set on the upper surface of the table.

3. The shoulder coil of claim 1 wherein the rigid housing is configured to receive a posterior side of the shoulder and the flexible coil assembly is configured to be positioned on an anterior side of the shoulder.

4. The shoulder coil of claim 1 wherein:
the rigid housing includes a first portion and a second portion,
the first portion is generally planar and is configured to be positioned between the shoulder of the patient and the table,
the first portion includes a first edge configured to be located between the shoulder of the patient and the table and a second edge configured to be located beyond the lateral surface of the shoulder,
the second portion extends away from the table for at least a portion of the lateral surface of the shoulder,
the second portion includes a first edge joined to the second edge of the first portion proximate to the table and a second edge distal from the table, and
the second portion is curved to extend between the superior surface of the shoulder and the lateral surface of the shoulder.

5. The shoulder coil of claim 4 wherein the first portion of the rigid housing has a first thickness and is tapered to a second thickness along at least a portion of the first edge of the first portion, wherein the second thickness is less than the first thickness.

6. The shoulder coil of claim 1 wherein the flexible coil assembly further includes a retaining member configured to retain the flexible coil in the second position.

7. The shoulder coil of claim 6 wherein the flexible coil assembly extends for a first distance from the rigid housing onto the shoulder of the patient and wherein the retaining member is a stiffening bar extending for at least a portion of the first distance.

8. The shoulder coil of claim 6 wherein the flexible coil assembly includes an outer periphery having a first edge mounted to the rigid housing and a second edge distal from the rigid housing and located over the shoulder during medical imaging and wherein the flexible coil assembly further includes a weighting member along at least a portion of the second edge.

9. The shoulder coil of claim 1, wherein the flexible coil assembly includes:
an inner housing;
an outer housing; and
a flexible substrate mounted between the inner and outer housings, wherein the plurality of second antennas are mounted to the flexible substrate.

10. The shoulder coil of claim 1, further comprising a strap configured to retain the lower support member in a desired orientation with respect to the patient.

11. A shoulder coil for use during medical imaging of a patient, comprising:
a rigid housing including a first portion and a second portion, wherein:
the first portion is configured to be positioned between a shoulder of the patient and a table of a magnetic resonance (MR) scanner,
the second portion extends from the first portion away from the table and is configured to be positioned between a superior surface of the shoulder and a lateral surface of the shoulder of the patient during medical imaging, and
a chamber is defined within the first and second portions;
a flexible coil assembly mounted to the second portion of the rigid housing, wherein the flexible coil assembly includes an inner housing, an outer housing, and a flexible substrate mounted between the inner and outer housing, and wherein the flexible coil assembly is movably positioned with respect to the rigid housing to allow the shoulder of the patient to be positioned on the rigid housing in a first position and to be placed adjacent to the shoulder of the patient in a second position, and
a plurality of antennas defining an antenna array within the shoulder coil, wherein a first set of the plurality of antennas are positioned within the chamber of the rigid housing and a second set of the plurality of antennas are mounted to the flexible substrate of the flexible coil assembly.

12. The shoulder coil of claim 11 further comprising a cable extending from one of the rigid housing and the flexible coil assembly to the MR scanner, wherein the cable transmits a signal generated by each of the plurality of antennas to the MR scanner.

13. The shoulder coil of claim 11 wherein the first portion of the rigid housing includes a substantially planar lower surface operative to set on an upper surface of the table and wherein at least part of the first set of the plurality of antennas are positioned within the first portion of the rigid housing such that each of the antennas is generally parallel to the upper surface of the table when the lower surface of the first portion is set on the upper surface of the table.

14. The shoulder coil of claim 11 wherein the rigid housing is configured to receive a posterior side of the shoulder and the flexible coil assembly is configured to be positioned on an anterior side of the shoulder.

15. The shoulder coil of claim 11 wherein:
the first portion includes a first edge configured to be located between the shoulder of the patient and the table and a second edge configured to be located beyond the lateral surface of the shoulder,
the second portion includes a first edge joined to the second edge of the first portion proximate to the table and a second edge distal from the table, and
the second portion is curved to extend between the superior surface of the shoulder and the lateral surface of the shoulder.

16. The shoulder coil of claim 15 wherein the first portion of the rigid housing has a first thickness and is tapered to a second thickness along at least a portion of the first edge of the first portion, wherein the second thickness is less than the first thickness.

17. The shoulder coil of claim 11 wherein the flexible coil assembly further includes a retaining member configured to retain the flexible coil in the second position.

18. The shoulder coil of claim 17 wherein the flexible coil assembly extends for a first distance from the rigid housing onto the shoulder of the patient and wherein the retaining member is a stiffening bar extending for at least a portion of the first distance.

19. The shoulder coil of claim 17 wherein the flexible coil assembly includes an outer periphery having a first edge mounted to the rigid housing and a second edge distal from the rigid housing and located over the shoulder during medical imaging and wherein the flexible coil assembly further includes a weighting member along at least a portion of the second edge.

20. The shoulder coil of claim 11 further comprising a strap configured to retain the rigid housing in a desired orientation with respect to the patient.
